(19) United States
(12) Patent Application Publication
(10) Pub. No.: US 2023/0130513 A1
(43) Pub. Date: Apr. 27, 2023

(54) Magnetic-Responsive Photosensitizer Nanoplatform and Uses Thereof

(71) Applicant: University of Maryland, Baltimore, Baltimore, MD (US)

(72) Inventors: Mary Anne Melo, Hanover, MD (US); Abdulrahman A. Balhaddad, Nottingham, MD (US); Michael D. Weir, Silver Spring, MD (US); Huakun Xu, Frederick, MD (US)

(73) Assignee: University of Maryland, Baltimore, Baltimore, MD (US)

(21) Appl. No.: 17/970,448

(22) Filed: Oct. 20, 2022

Related U.S. Application Data
(60) Provisional application No. 63/270,193, filed on Oct. 21, 2021.

(51) Int. Cl. A61K 9/107; A61K 9/51; A61K 9/00; A61P 31/04; A61P 1/02; A61Q 11/00; A61K 41/00; A61K 33/26; A61K 8/06; A61K 8/19; A61K 8/49; A61N 5/06

(52) U.S. Cl. CPC A61K 9/1075; A61K 9/5115; A61K 9/0009; A61P 31/04; A61P 1/02; A61Q 11/00; A61K 41/0057; A61K 41/0052; A61K 33/26; A61K 8/068; A61K 8/19; A61K 8/49; A61N 5/062; A61N 5/0624; A61K 2800/81; A61K 2800/47; A61K 2800/26; A61N 2005/0606

(57) ABSTRACT

Provided herein are magnetically-responsive, photosensitizer based antimicrobial microemulsions and photodynamic nanoplatforms in which a photosensitizer functionally associated with a plurality of superparamagnetic iron oxide nanoparticles are encapsulated. Also provided are methods and processes utilizing the antimicrobial microemulsions and photodynamic nanoplatforms to treat an oral disease, to reduce a microbial population in a pathogenic oral biofilm and to improve the efficacy of a photosensitizer during an antimicrobial photodynamic therapy treatment of an oral disease.

### CROSS-REFERENCE TO RELATED APPLICATIONS

This non-provisional patent application claims benefit of priority under 35 U.S.C. § 119(e) of provisional application U.S. Ser. No. 63/270,193, filed Oct. 21, 2021, the entirety of which is hereby incorporated by reference.

### BACKGROUND OF THE INVENTION

### Field of the Invention

The present invention relates to the fields of dentistry, oral diseases, and drug delivery systems. More particularly, the present invention relates to microemulsions as magnetic responsive nanoplatforms for the delivery of formulations to pathogenic microbes in the oral environment during photodynamic therapy.

### Description of the Related Art

Biofilm control within the complexity of the intraoral environment is a challenging task (1). The continuing rise in antibiotic resistance, the ongoing problem with patient compliance, and the difficulty in eradicating biofilms demand the use of combinatorial strategies (2). Biofilm formation is an important virulence mechanism of oral pathogens (3). Oral dysbiotic biofilms trigger major oral diseases, such as dental caries, periodontitis, and endodontic infections (4). Caries-related pathogens can secrete a mixture of polysaccharides to construct and maintain a structured multicellular bacterial community into biofilm to survive and grow (5). The extracellular matrix resists antimicrobial agents from reaching targeted microbial cells by diffusion limitation. This barrier for tolerance against antimicrobial agents has been recognized (6,7).

Antimicrobial photodynamic therapy (aPDT) is a promising adjunctive method for modulating pathogenic oral biofilms that is well supported by a plethora of literature (7-10). Antimicrobial photodynamic therapy uses non-toxic dyes called photosensitizers (PS) that can be excited by harmless visible light to produce cytotoxic reactive oxygen species (ROS) (5). Antimicrobial photodynamic therapy involves a multi-stage process, including topical photosensitizer administration, light irradiation, and interaction of the excited state with the surrounding oxygen (11). For oral biofilm-associated diseases, especially dental caries, antimicrobial photosensitizers based on cationic phenothiazine dye, toluidine blue O (TBO), have been well studied and mediated expressive photodynamic results (11,12). However, some difficulties still need to be improved in antimicrobial photodynamic therapy, such as specific targeting and biological compatibility.

Clinically, antimicrobial photodynamic therapy performance is confined to subsidiary outcomes without clinically relevant reduction greater than 3-log (>99.9%) (11,13,14). The conceivable explanations for the resistance of biofilms are limited diffusion and interaction of antimicrobial agents through the biofilm, altered levels of metabolic activity within the biofilm, and genetic adaptation (5). In addition, the transport and infiltration of the photosensitizer into the biofilm core structure is an essential step in optimizing the antimicrobial photodynamic therapy performance (15).

Several antibiofilm strategies have been explored to modulate biofilm formation and development (16). In addition, targeting strategies to break the biofilm barrier or enhance the infiltration of antibacterial agents are being increasingly explored. Therefore, the rational design of photosensitizer-based nanoplatforms to overcome the above key obstacles for achieving potent aPDT is of great significance for managing oral infections.

The prior art is deficient in antimicrobial photodynamic therapies utilizing a magnetic-responsive photosensitizer-based nanoplatform. The present invention fulfills this long-standing need and desire in the art.

### SUMMARY OF THE INVENTION

The present invention is directed to an antimicrobial microemulsion. The microemulsion comprises a plurality of superparamagnetic iron oxide nanoparticles (SPIONs) and a photosensitizer in a functional association with the plurality of SPIONs.

The present invention is further directed to a method for treating an oral disease in a subject in need thereof. In this method, the antimicrobial microemulsion described herein is applied to an oral area of interest in the subject. The plurality of superparamagnetic iron(II) oxide nanoparticles and the photosensitizer in the functional association therewith are irradiated during an application of a magnetic field to the superparamagnetic iron(II) oxide nanoparticles. The plurality of superparamagnetic iron(II) oxide nanoparticles and the photosensitizer are targeted to the oral area of interest via the magnetic field, thereby treating the oral disease in the subject.

The present invention is directed further to a magnetic-responsive photodynamic nanoplatform. The magnetic-responsive photodynamic nanoplatform comprises, in a microemulsion, a plurality of superparamagnetic iron(II) oxide nanoparticles and an organic photosensitizer functionally associated with the plurality of superparamagnetic iron(II) oxide nanoparticles.

The present invention is directed further still to a process for decreasing a microbial population in a pathogenic oral biofilm. In this method, the oral biofilm is contacted with the microemulsion comprising the magnetic-responsive photodynamic nanoplatform described herein. A photodynamic therapy is applied to the plurality of superparamagnetic iron(II) oxide nanoparticles and the organic photosensitizer functionally associated therewith in the microemulsion. A magnetic field is applied to the superparamagnetic iron(II) oxide nanoparticles during the photodynamic therapy to target the plurality of superparamagnetic iron(II) oxide nanoparticles and the organic photosensitizer to the microbial population of the pathogenic oral biofilm, where the plurality of superparamagnetic iron(II) oxide nanoparticles and the organic photosensitizer are pharmacologically effective to decrease the microbial population therein.

The present invention is directed further still to a method for improving the efficacy of a photosensitizer during an antimicrobial photodynamic therapy (aPDT) treatment of oral diseases. In this method, a photosensitizer and a plurality of superparamagnetic iron(II) oxide nanoparticles are encapsulated in a microemulsion whereby the photosensitizer is functionally associated with the plurality of superparamagnetic iron(II) oxide nanoparticles. The photosensitizer and the superparamagnetic iron(II) oxide nanoparticles are irradiated to activate the same during an application of a magnetic field thereto, whereby the magnetic field directs the superparamagnetic iron(II) oxide nanoparticles with the functionally associated photosensitizer to a pathogenic microbe causing the oral disease, thereby improving the efficacy of the photosensitizer during the aPDT treatment of the oral disease.

Other and further aspects, features, benefits, and advantages of the present invention will be apparent from the following description of the presently preferred embodiments of the invention given for the purpose of disclosure.

### BRIEF DESCRIPTION OF THE FIGURES

The appended drawings have been included herein so that the above-recited features, advantages, and objects of the invention will become clear and can be understood in detail. These drawings form a part of the specification. It is to be noted, however, that the appended drawings illustrate preferred embodiments of the invention and should not be considered to limit the scope of the invention.

FIGS. 1A-1B are schematic drawings showing the microemulsions' synthesis. FIG. 1A shows a high ultrasonication method was used to synthesize the microemulsions. FIG. 1B shows a phase diagram of the synthesized microemulsions. 40% of the microemulsions were composed of surfactants, while the remaining were composed of an equal amount of eucalyptus oil and water.

FIGS. 2A-2E shows TBO and superparamagnetic iron(II) oxide nanoparticle characterization. FIG. 2A is the FT-IR spectrum of the TBO photosensitizer showing the fingerprint regions. FIG. 2B shows the maximum peak absorption at 594 and 632 nm overlapping with the light-emitting diode (LED) spectrum. FIG. 2C is a histogram graph that shows the size range of the superparamagnetic iron oxide nanoparticles. FIGS. 2D-2E are transmission electron microscopy (TEM) images showing the distribution of superparamagnetic iron(II) oxide nanoparticles inside the microemulsions.

FIGS. 3A-3D shows TBO and superparamagnetic iron(II) oxide nanoparticles magnetization. FIG. 3A is a magnetization curve of the superparamagnetic iron oxide nanoparticles at room temperature. FIG. 3B shows the microscopic magnetic field around a cylindric magnet with 1T (diameter of 10 mm and length of 30 mm). FIG. 3C shows the cylindric magnet with a magnetite nanoparticle locating at 5 mm above the magnet. Blue lines mark the position of the magnet. The nanoparticle locates at the center of the marked red region and cannot be seen by naked eyes. FIG. 3D shows a zoomed in magnetic field of the marked region in FIG. 3C.

FIGS. 4A-4C shows fibroblast viability. FIG. 4A shows the response of the human gingival fibroblasts toward the superparamagnetic iron oxide nanoparticles with and without TBO (mean±sd; n=9). FIG. 4B shows the response of the human gingival fibroblasts toward MagTBO microemulsions at 1:1 ratio (mean±sd; n=9). *Asterisks indicate significant differences compared to the control (p<0.05). FIGS. 4C-4F are representatives live/dead images of human gingival fibroblasts treated with, respectively, Control, 5% MagTBO, 2.5% MagTBO, and TBO alone (n=3).

FIGS. 5A-5D illustrates the antibiofilm efficacy of the MagTBO Platform in the photodynamic process. FIG. 5A shows the colony-forming units (CFUs) of the core components of the microemulsion, FIG. 5B shows the MagTBO microemulsions, TBO alone without light activation, and FIG. 5C shows the MagTBO microemulsions and TBO alone with light activation (mean±sd; n=9). Values indicated by different letters are statistically different from each other (p<0.05). FIG. 5D shows the Streptococcus mutans colonies on brain-heart infusion (BHI) agar. The use of the 2.5% and 5% MagTBO microemulsions with magnetic field completely eradicated the S. mutans biofilms.

FIGS. 6A-6H are representative live/dead images of biofilms. FIG. 6A is a control image, FIG. 6B is antimicrobial photodynamic therapy with TBO alone, FIGS. 6C-6D are 1% MagTBO microemulsions with and without magnetic field, FIGS. 6E-6F are 2.5% MagTBO microemulsions with and without magnetic field and FIGS. 6G-6H are 5% MagTBO microemulsions with and without magnetic field. Live bacteria were stained green, and compromised bacteria were stained red.

FIGS. 7A-7D are scanning electron microscopy (SEM) images of the S. mutans biofilms grown over the borosilicate glass slabs (n=3). FIG. 7A shows a biofilm with no treatment, FIG. 7B shows a biofilm treated using TBO, FIG. 7C shows a biofilm treated using 2.5% MagTBO, and FIG. 7D shows a biofilm using 2.5% MagTBO with a magnetic field.

FIGS. 8A-8C shows colony-forming units (CFUs) of the saliva-derived biofilms subjected to different treatments and dentin microhardness following the biofilm challenge. FIG. 8A is a schematic drawing demonstrating the design of the saliva-derived biofilm assay. The biofilm was grown over the slabs for seven days, and then the slabs were transferred to perform colony-forming units, Vicker's microhardness, scanning electron microscopy, and confocal fluorescence microscopy. FIG. 8B shows the CFUs of the total microorganisms, total streptococci, mutans streptococci, and total lactobacilli (mean±sd; n=6). Values indicated by different letters are statistically different from each other (p<0.05). FIG. 8C shows the reduction in dentin hardness in percentage after exposing the dentin slabs to 7-day biofilms (mean±sd; n=6). Values indicated by different letters are statistically different from each other (p<0.05).

FIGS. 9A-9L are confocal images of the saliva-derived biofilms grown over the dentin slabs (n=3). FIGS. 9A-9C are biofilms with no treatment, FIGS. 9D-9F are biofilms treated using TBO, FIGS. 9G-9I are biofilms treated using 2.5% MagTBO, and FIGS. 9J-9L are biofilms treated using 2.5% MagTBO with the magnetic field.

FIGS. 10A-10D are scanning electron microscopy images of the saliva-derived biofilms grown over the dentin slabs (n=3). FIG. 10A is the biofilm with no treatment, FIG. 10B is the biofilm treated using TBO, FIG. 10C is the biofilm treated using 2.5% MagTBO, and FIG. 10D is the biofilm treated using 2.5% MagTBO with the magnetic field.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements or steps but not the exclusion of any other item, element or step or group of items, elements or steps unless the context requires otherwise. Similarly, “another” or “other” may mean at least a second or more of the same or different claim element or components thereof.

As used herein, “photosensitizer-based nanoplatform”, “magnetic-responsive photodynamic nanoplatform”, “nanoplatform”, “antimicrobial microemulsion”, and “MagTBO nanoplatform” are used interchangeably.

As used herein, “contacting” refers to any suitable method of bringing the superparamagnetic iron oxide nanoparticles and the photosensitizer comprising the nanoplatforms or antimicrobial microemulsions described herein into contact with a pathogenic oral biofilm or pathogenic microbe associated with an oral disease. For in vivo applications, any known method of administration within the oral cavity is suitable. In vitro, this is achieved by exposing the pathogenic oral biofilm or pathogenic microbe to the superparamagnetic iron oxide nanoparticles and the photosensitizer in a suitable medium.

As used herein, “pharmacologically effective” refers to an amount or concentration of the superparamagnetic iron oxide nanoparticles and the photosensitizer in the microemulsion that results in an improvement or remediation in the oral disease or in a decrease in or a reduction up to elimination of the microbial population of a pathogenic oral biofilm. A person having ordinary skill in this art would understand that the pharmacologically effective amount may improve the patient's or subject's condition, but may not be a complete cure of the oral disease or effect a complete elimination of the microbial population in the oral biofilm.

As used herein, the term “subject” refers to any target or recipient of the treatments utilizing the magnetic-responsive photodynamic nanoplatforms or antimicrobial microemulsions provided herein.

In one embodiment of the present invention there is provided an antimicrobial microemulsion, comprising a plurality of superparamagnetic iron oxide nanoparticles; and a photosensitizer in a functional association with the plurality of superparamagnetic iron(II) oxide nanoparticles.

In this embodiment, the superparamagnetic iron oxide nanoparticles may be superparamagnetic iron(II) oxide nanoparticles. Also in this embodiment a representative photosensitizer is toluidine blue O (TBO). The antimicrobial microemulsion may comprise a magnetic-responsive photodynamic nanoplatform configured for targeted delivery of the plurality of superparamagnetic iron(II) oxide nanoparticles and the photosensitizer.

In a related embodiment, the present invention provides a pharmaceutical composition comprising the antimicrobial microemulsion, as described supra.

In another embodiment of the present invention, there is provided a method for treating an oral disease in a subject in need thereof, comprising applying the antimicrobial microemulsion, as described supra, to an oral area of interest in the subject; irradiating the plurality of superparamagnetic iron(II) oxide nanoparticles and the photosensitizer in the functional association therewith during an application of a magnetic field to the superparamagnetic iron(II) oxide nanoparticles; and targeting, via the magnetic field, the plurality of superparamagnetic iron(II) oxide nanoparticles and the photosensitizer to the oral area of interest, thereby treating the oral disease in the subject.

In an aspect of this embodiment, the irradiating step may comprise applying an antimicrobial photodynamic therapy. In this embodiment and aspect, thereof the oral disease may be selected from the group consisting of at least one of caries, periodontitis, peri-implantitis, and an endodontic infection.

In yet another embodiment of the present invention, there is provided a magnetic-responsive photodynamic nanoplatform, comprising, in a microemulsion, a plurality of superparamagnetic iron(II) oxide nanoparticles; and an organic photosensitizer functionally associated with the plurality of superparamagnetic iron(II) oxide nanoparticles. In this embodiment, the organic photosensitizer may be toluidine blue O (TBO).

In yet another embodiment of the present invention, there is provided a process for decreasing a microbial population in a pathogenic oral biofilm, comprising contacting the oral biofilm with the microemulsion comprising the magnetic-responsive photodynamic nanoplatform, as described supra; applying a photodynamic therapy to the plurality of superparamagnetic iron(II) oxide nanoparticles and the organic photosensitizer functionally associated therewith in the microemulsion; and applying a magnetic field to the superparamagnetic iron(II) oxide nanoparticles during the photodynamic therapy to target the plurality of superparamagnetic iron(II) oxide nanoparticles and the organic photosensitizer to the microbial population of the pathogenic oral biofilm, where the plurality of superparamagnetic iron(II) oxide nanoparticles and the organic photosensitizer are pharmacologically effective to decrease the microbial population therein.

In an aspect of this embodiment, the step of applying the photodynamic therapy may comprise irradiating the plurality of superparamagnetic iron(II) oxide nanoparticles and the organic photosensitizer functionally associated therewith. In this embodiment and aspect thereof the photodynamic therapy is an antimicrobial photodynamic therapy. In this embodiment and aspect thereof, the microbial population in the pathogenic oral biofilm may be a single pathogenic species biofilm or a multispecies pathogenic biofilm. In addition the microbial population may comprise at least one oral pathogenic microbe selected from the group consisting of Streptococcus mutans, Enterococcus faecalis, and Porphyromonas gingivalis.

In yet another embodiment of the present invention, there is provided a method for method for improving the efficacy of a photosensitizer during an antimicrobial photodynamic therapy treatment of an oral disease, comprising encapsulating a photosensitizer and a plurality of superparamagnetic iron(II) oxide nanoparticles in a microemulsion whereby the photosensitizer is functionally associated with the plurality of superparamagnetic iron(II) oxide nanoparticles; and irradiating the photosensitizer and the superparamagnetic iron(II) oxide nanoparticles to activate the same during an application of a magnetic field thereto, whereby the magnetic field directs the superparamagnetic iron(II) oxide nanoparticles with the functionally associated photosensitizer to a pathogenic microbe causing the oral disease, thereby improving the efficacy of the photosensitizer during the aPDT treatment of the oral disease.

In an aspect of this embodiment, the microemulsion encapsulating the superparamagnetic iron(II) oxide nanoparticles and the photosensitizer functionally associated therewith may comprise a photodynamic nanoplatform. In this embodiment and aspect thereof the photosensitizer may be toluidine blue O (TBO). In this embodiment and aspect thereof, the oral disease may be selected from the group consisting of at least one of caries, periodontitis, peri-implantitis, and an endodontic infection. In addition, the pathogenic microbe may be selected from the group consisting of at least one of Streptococcus mutans, Enterococcus faecalis, and Porphyromonas gingivalis.

Provided herein are magnetic-responsive, photosensitizer-based nanoplatforms and antimicrobial microemulsions which enable an enhanced antimicrobial effect via association with iron(II) oxide or ferrous oxide nanoparticles (Fe₂O₃) and magnetic field navigation. The magnetic-responsive photodynamic nanoplatform of the present invention (MagTBO) was constructed by assembling TBO and superparamagnetic iron oxide nanoparticles using a continuous microemulsion (F1).

In the nanoplatform, the synthesized microemulsions improved the stability, dispersity, and biocompatibility of nanoparticles and enhanced the antimicrobial action of the TBO photosensitizer. Moreover, the exposure to external magnetic forces allows the motion of the therapeutic agents toward deep sites inside the biofilms, providing potential disruption of the self-produced extracellular polysaccharide biofilm matrix and biofilm reduction. The present invention demonstrates that antimicrobial photodynamic therapy via the MagTBO nanoplatform, guided by magnetic force, are pharmacologically effective to treat biofilms formed by a major pathogen associated with dental caries and other oral diseases.

The following examples are given for the purpose of illustrating various embodiments of the invention and are not meant to limit the present invention in any fashion.

### Example 1

### Methods and Materials

### Spectra of TBO and LED

The TBO photosensitizer has an absorption peak between 594 and 632 nm, assessed via ultraviolet-visible optical absorption spectrometry (SpectraMax M5, Molecular Devices, Sunnyvale, Calif.). The TBO spectrum also was assessed using Fourier-transform infrared spectroscopy (FTIR) (Nicolet 6700, Thermo Fisher Scientific, Waltham, Mass.). A light-emitting diode (LED; photoactivated disinfection (PADLight-F3WW, Beijing, China) with a narrow spectrum emission ranging from 664 to 670 nm was used for the light activation. The diameter of the LED light tip was 6 mm. A power meter Lasermate (Coherent Inc., Santa Clara, Calif.) at a 2 mm distance from the LED light tip was used to measure the peak power. The irradiation of 5 min was associated with an energy density of 180 J/cm2, which was calculated using the following equation:

$$Energy density ( \frac{J}{{cm}^{2}} ) = Fluency = \frac{power density ( W ) \times time ( sec )}{A ( {cm}^{2} )} ,$$

where power density is:

$$Power density = \frac{light intensity ( W )}{A ( {cm}^{2} )} .$$

### Synthesis, Characterization, and Magnetization of the Magnetic Nanoparticles

Superparamagnetic iron(II) oxide nanoparticles were synthesized via chemical co-precipitation as described by Sun et al. (17). Briefly, 10 mL of deionized water was used to dissolve 0.2 g of polyglucose-sorbitol-carboxymethyl-ether (PSC). Then, 15 mL of water containing 0.06 g of FeCl₃ and 0.03 g of FeCl₂ was added. The mixture was cooled to 5° C., and 1 g of 28% ammonium hydroxide was added and stirred for 2 min. Finally, the mixture was heated at 80° C. for 1 h and purified using a 100 kDa membrane with five cycles.

The size and distribution of superparamagnetic iron(II) oxide nanoparticles were examined by transmission electron microscopy (TEM, FEI Tecnai T20, Hillsboro, Oreg., USA) with the Software Imaging System CCD camera (Gatan UltraScan 1000, Milwaukee, Wis., USA). The images were captured using 80 kV and magnifications of 26,000× and 42,000×. The samples were examined in the microemulsion form. ImageJ software was applied to measure the size of the nanoparticles. The histogram illustrated the size distribution using Sigma Plot software (SYSTAT, Chicago, Ill., USA). A semi-analytical solution with 98% accuracy (Ortner2020SoftwareX, Furlani1994IEEE) was used to calculate the magnetic fields. The calculation used a neodymium magnet in a cylindric shape and a diameter of 10 mm, length of 30 mm, and T1 magnetic B-field strength. A Fe₃O₄ nanoparticle with an average diameter of 8.4 nm was chosen and treated as magnetic dipoles in the calculation. The magnetic dipole moment of the nanoparticle was derived from magnetization curves.

### Cell Cytotoxicity Assay

Human gingival fibroblasts (HGF, ScienCell, San Diego, Calif., USA) were cultured using fibroblast medium (FM) supplemented with 2% fetal bovine serum, 1% fibroblast growth supplement, 100 IU/mL penicillin, and 100 IU/mL streptomycin (18 76). When the viability of the cells was above 90%, the cells were seeded in the wells of a 96-well plate (5,000 cells per well) and incubated for 24 h. In the following day, the medium was removed, and different concentrations of superparamagnetic iron(II) oxide nanoparticles (0.25, 0.5, 1, 1.5, 2, 2.5, and 5 wt. %) nanoparticles with and without 100 μg/mL of TBO photosensitizer were dissolved in the fibroblast medium and incubated in contact with the cells for 24 h. The synthesized microemulsions containing the different concentrations of superparamagnetic iron(II) oxide nanoparticles (0.25, 0.5, 1, 1.5, 2, 2.5, and 5 wt. %) were also tested. All the microemulsions were diluted with the fibroblast medium at a 1:1 ratio. After 24 h of incubation at 37° C. with 5% CO₂, the cells were washed with phosphate-buffered saline (PBS), and cell counting Kit-8 (CCK-8, Dojindo, Rockville, Md., USA) was used to evaluate the cell viability. The absorbance was read at 450 nm using a spectrophotometer (SpectraMax M5, Molecular Devices, Sunnyvale, Calif., USA). For the live/dead assay, the fibroblast cells were seeded in the wells of a 24-well plate (40,000 cells per well). The medium was changed daily to allow the viability to be above 90%. Microemulsions containing 5 and 2.5% were added to a fresh culture medium at the ratio of 1:1 and incubated with the cells for one day. Wells treated with TBO alone or with no treatment were used as controls. After one day of incubation, the old medium was removed, cells were washed with PBS, and LIVE/DEAD™ Cell Imaging Kit (Invitrogen, Frederick, Md., USA) was used to stain the cells. The cells were visualized using an inverted epifluorescence microscope (Eclipse TE2000-S, Nikon, Melville, N.Y., USA).

### Optimization and Evaluation of MagTPO Microemulsions

Three microemulsions were selected for further investigations (1, 2.5, and 5 wt. %). Microemulsions were tested for thermodynamic stability via centrifugation stress at 5,000 rpm for 2 h at 4° C., and freeze-thaw stress consisting of three complete cycles. Each cycle consisted of placing the microemulsion for 24 h at −20° C. followed by another 24 h at 4° C. The microemulsions were observed for any physical changes of phase separation (19,20). For long-term stability, microemulsions were kept in the dark at room temperature and then examined at 1, 3, and 6 months for physical changes or phase separation (21,22). The pH of the synthesized microemulsions was tested using a digital pH meter (accuracy±5%; Accumet XL25, Thermo Fisher Scientific, Waltham, Mass., USA). Before the pH measurement, the pH meter was calibrated using commercial standard buffer solutions of pH 4, pH 7, and pH 10 at room temperature. For the absorbance evaluation, the upper third of the synthesized microemulsions was investigated over time using ultraviolet-visible optical absorption spectrometry (SpectraMax M5, Molecular Devices, Sunnyvale, Calif., USA). A large change in the absorbance value indicates that the microemulsion may suffer from unbalanced distribution of the nanoparticles or some physical changes. The density of each microemulsion was calculated following the equation:

$$Density = \frac{Mass}{Volume} .$$

Photodynamic Treatment Via MagTBO Against Streptococcus mutans Biofilms

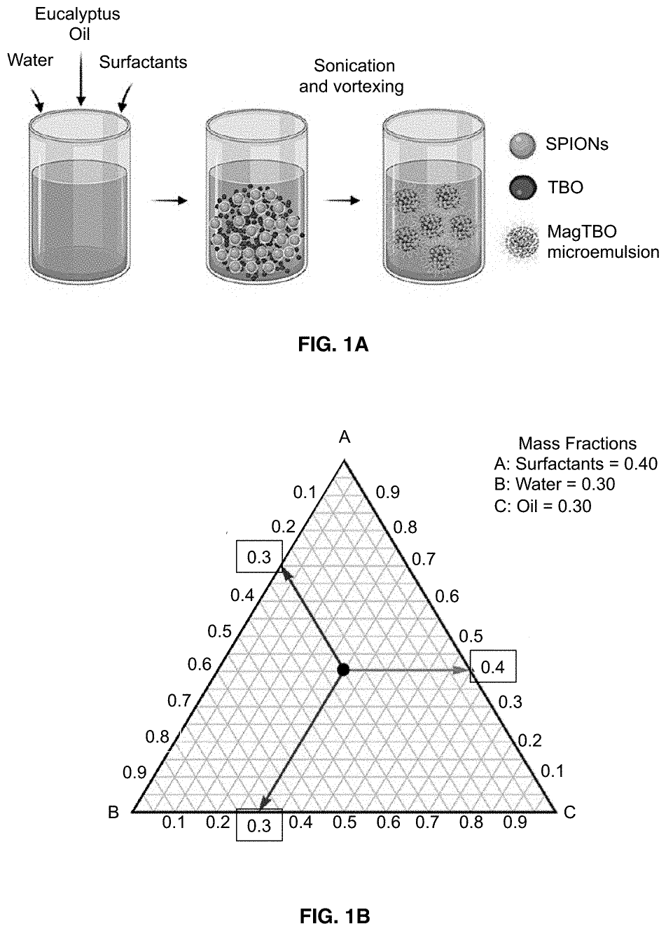

S. mutans UA159 obtained from the American Type Culture Collection (ATCC, Manassas, Va., USA) were grown overnight using brain heart infusion (BHI) broth (Sigma-Aldrich, St. Louis, Mo., USA) at 37° C. and 5% CO₂ incubator. The optical density of the S. mutans was modified to 1×10⁸ colony-forming units (CFU)/mL (OD₆₀₀=0.9) and diluted 1:20 with BHI broth supplemented with 2% of sucrose (12). 200 μL of the diluted overnight culture was placed inside the wells of a black 96-well plate for 24 h. The media was changed after 24 h and 48 h of biofilm growth was continued. The biofilm was washed with 0.9% saline and irradiated with 100 μg/mL of TBO and MagTBO microemulsions containing 1, 2.5, and 5% of SPIONs with and without magnetic field (neodymium magnet, 0.4 Tesla; pull force=601b5). The TBO or the microemulsions were left for 1 min and then irradiated for 5 minutes to deliver an energy density of 180 J/cm² and light intensity of 180 mW. The magnetic field was applied at a 10 mm distance below the biofilm during the entire period of the incubation and irradiation times (FIG. 1C). Untreated wells and wells treated with glycerol, essential oil, and polysorbate 20 were used as controls.

After each treatment, the biofilms were washed by placing 200 μL of 0.9% saline inside the well, and then, the 0.9% saline was gently aspirated. Next, another 200 μL of saline was placed inside the well, and the biofilm was gently scraped to remove the biofilm cells (23). Then, the biofilm cells were resuspended with the saline to perform serial dilutions (1:10, 1:100, 1:1000, 1:10 000, and 1:100 000) using BHI agar. The BHI agar plates were incubated for 48 h at 37° C. in a 5% CO2 incubator and then counted using a colony counter. Considering the dilution factor, the CFUs/mL were calculated and estimated to measure the number of colonies per milliliter. Two wells were randomly selected for Live/Dead staining of the biofilms to be washed with 0.9% saline and then stained with the BacLight live/dead kit (Molecular Probes, Eugene, Oreg.). A combination of 2.5 μM SYTO 9 and 2.5 μM propidium iodide at a ratio of 1:1 was created to treat the cells for 10 min and observed using an inverted epifluorescence microscope (Eclipse TE2000-S, Nikon, Melville, N.Y.).

Scanning Electron Microscopy (SEM) Imaging of the S. mutans Biofilms

To investigate the effect of the MagTBO microemulsion against the biofilm thickness and morphology, the 2.5% MagTBO effect against a 48-h S. mutans biofilms was further analyzed using Scanning Electron Microscope. S. mutans biofilms were grown over borosilicate glass slabs (VWR, Radnor, Pa., USA) for 48 h. Then, the biofilms were washed with PBS and fixed using 3% formaldehyde. On the following day, the biofilms were dried using ethanol dilutions followed by 100% hexamethyldisilazane. The biofilms were then sputter-coated with platinum and capture using SEM (Quanta 200, FEI Company, Hillsboro, Oreg., USA).

Photodynamic Treatment with MagTBO Against Saliva-Derived Biofilms

Saliva samples were collected from ten healthy individuals with no active carious lesions or history of antibiotics in the last three months. The participants were instructed not to brush their teeth 24 h and not eat or drink two h before the collection. The collected saliva samples were mixed to create a homogenous and complex bacterial composition. The use of saliva as inoculum was approved by the University of Maryland Baltimore Institutional Review Board (HP-00050407). The collected saliva was mixed with glycerol (70:30) and stored at −80° C. This model was used in previous studies to form thick and mature multispecies biofilm (24-26).

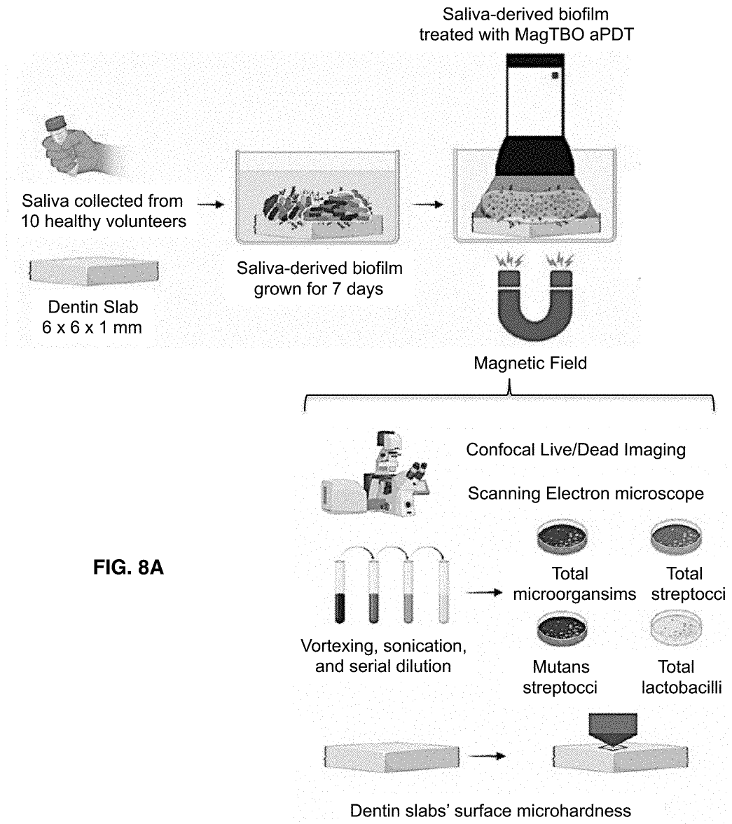

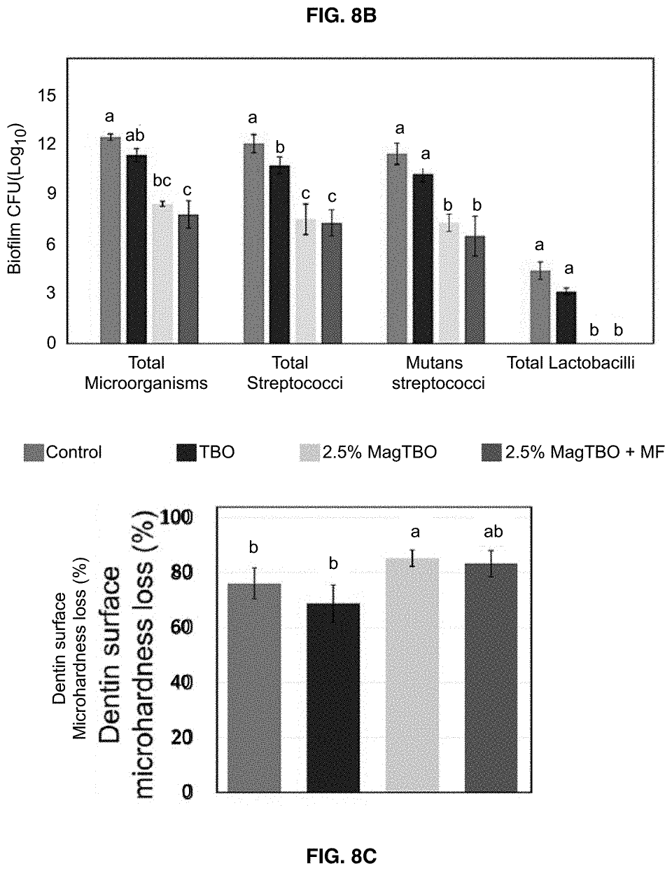

Dentin slabs (6×6×1 mm) were used as substrates to grow the biofilm (FIG. 8A). The slabs were prepared from radicular dentin of extracted teeth using a diamond saw with water as coolant (Isomet, Buehler, Lake Bluff, Ill., USA). The collection and the use of extracted teeth were approved by the University of Maryland Baltimore Institutional Review Board. The slabs were polished using sandpapers with the grit of #600, 1200, 2400, and 4000, consecutively, then screened based on their Vicker's microhardness value. The microhardness of the slabs was measured via Vicker's indentation (HMV II, Shimadzu Corporation, Kyoto, Japan) by calculating the average of five different indentations (25 g load for 10 s) per slab (27). Slabs with a microhardness mean variation of more than 15% compared to the microhardness mean of all slabs were excluded.

To inoculate the saliva-derived biofilm model, the saliva-glycerol stock was mixed with McBain artificial saliva (1:50) and placed inside the wells of a 24-well plate containing the dentin slabs. The components of the McBain growth medium were as the following: mucin (Type II, porcine, gastric), 2.5 g/L; bacteriological peptone, 2.0 g/L; tryptone, 2.0 g/L; yeast extract, 1.0 g/L; NaCl, 0.35 g/L, KCl, 0.2 g/L; CaCl₂, 0.2 g/L; 50 mM pipes, 15 g/L; hemin, 0.001 g/L; vitamin K1, 0.0002 g/L, at pH 7, and 0.2% of sucrose (28 81). Each dentin slab was immersed with 1.5 mL of the inoculum. The growth medium was replaced after 8 and 24 h, and then daily till seven days of biofilm growth. Later, the dentin slabs were subjected to antimicrobial photodynamic therapy treatment via 100 μg/mL of TBO, 2.5% MagTBO, 2.5% MagTBO with the magnetic field, and control with no treatment. The slabs were transferred to a vial containing 1 mL CPW solution, and the biofilms were harvested by vertexing and sonication. The following agar plates were prepared to enumerate the grown species:

a. Tryptic soy blood agar to count the total microorganisms.

b. Mitis salivarius agar (MSA) supplemented with potassium tellurite to count the total streptococci.

c. MSA supplemented with potassium tellurite and 0.2 units of bacitracin per mL to count mutans streptococci.

d. Rogosa agar to count the total lactobacilli.

The bacterial suspensions were serially diluted and plated in the agar plates and incubated for 48 h at 37° C. in 5% CO₂, except for the rogosa plates where the incubation was continued for 4 to 5-day.

### Dentin Surface Microhardness Following the 7-Day Saliva-Derived Biofilms

After biofilm collection, the Vicker's microhardness of dentin slabs was assessed as described (27). The amount of microhardness reduction was measured in percentages (%) as

$$dentin surface microhardness loss ( \% ) = \frac{surface hardness at baseline - surface hardness after biofilm collection}{surface hardness at baseline} \times 100.$$

### Confocal Fluorescence & Scanning Electron Microscopy Imaging of the Saliva-Derived Biofilms

The saliva-derived biofilm was initiated over dentin slabs, as described in the previous section. The dentin slabs were subjected to antimicrobial photodynamic therapy treatment via 100 μg/mL of TBO, 2.5% MagTBO, 2.5% MagTBO with the magnetic field, and control with no treatment. Then, the dentin slabs were prepared for confocal microscopy and SEM as explained with the S. mutans biofilms.

### Statistical Analysis

Shapiro-Wilk test was used to evaluate the data normality and distribution. Then, one-way ANOVA was used to evaluate the cytotoxicity of the microemulsions, and two-way ANOVA and Tukey's post hoc tests were used to analyze the effect of the magnetic nanoparticles and the effect of the magnetic field against the biofilms. All tests were conducted using the statistical software package Sigma Plot 12.0 (SYSTAT, Chicago, Ill., USA), and the statistical significance was set at p<0.05.

### Example 2

### Results

### Preparation of MagTBO Nanoplatform Via Microemulsion

MagTBO microemulsions were synthesized using a high-ultrasonication method (18,19). TBO (100 μg/mL; #T3260, Sigma-Aldrich, St. Louis, Mo.) and different mass fractions of Fe2O3 nanoparticles (0.25, 0.5, 1, 1.5, 2, 2.5, and 5 wt %) were added to a mixture of distilled water, eucalyptus oil (Spectrum, New Brunswick, N.J.), polysorbate 20 (AmericanBio, Canton, Mass.), and glycerol (FIG. 1A). An ultrasonic cell disruptor with an ultrasonication intensity of 40% was used to synthesize the microemulsions, as it is shown in FIG. 1A. The sonication was performed for 24 min using multiple cycles of 10 s of active sonication and 2 s of rest. Each microemulsion contained 40% of polysorbate 20 and glycerol at a ratio of 3:2 as surfactants, despite the used concentration of superparamagnetic iron(II) oxide nanoparticles. While the remaining were composed of an equal amount of eucalyptus oil and water (FIG. 1B).

### TBO and SPIONs Characterization and Magnetization

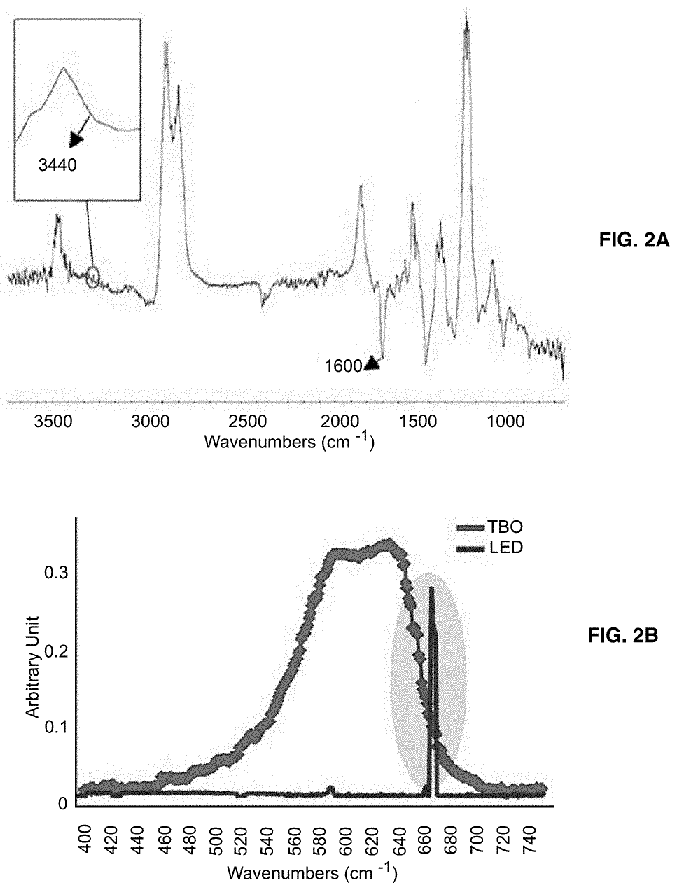

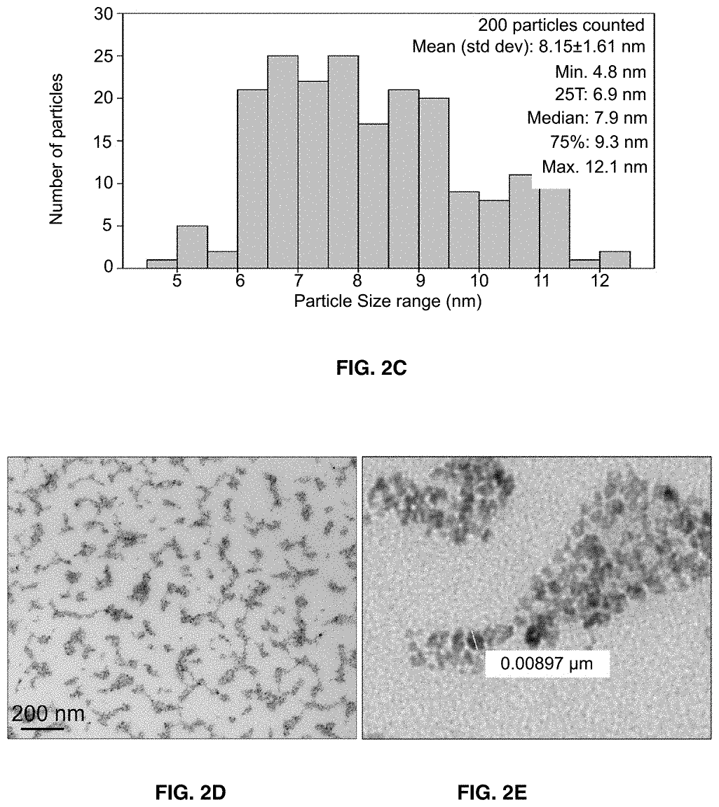

FIG. 2A demonstrates the FTIR spectrum of the TBO photosensitizer, which is characterized by the aromatic ring's band (≈1600 cm-1) and the primary amine stretch band (≈3340 cm-1). In FIG. 2B, the absorption band of the TBO between 550 and 650 nm is shown with two peaks at 594 and 632 nm. The spectrum of the TBO is overlapped with the spectrum of the LED light source. FIG. 2C illustrates the size distribution of superparamagnetic iron(II) oxide nanoparticles, which ranged between 4.8 and 12.1 nm with a mean size of 8.15±1.61 nm. The transmission electron microscopy (TEM) images of superparamagnetic iron(II) oxide nanoparticles within the microemulsion are shown in FIGS. 2D-2E. The nanoparticles are distributed homogeneously within the microemulsion structure.

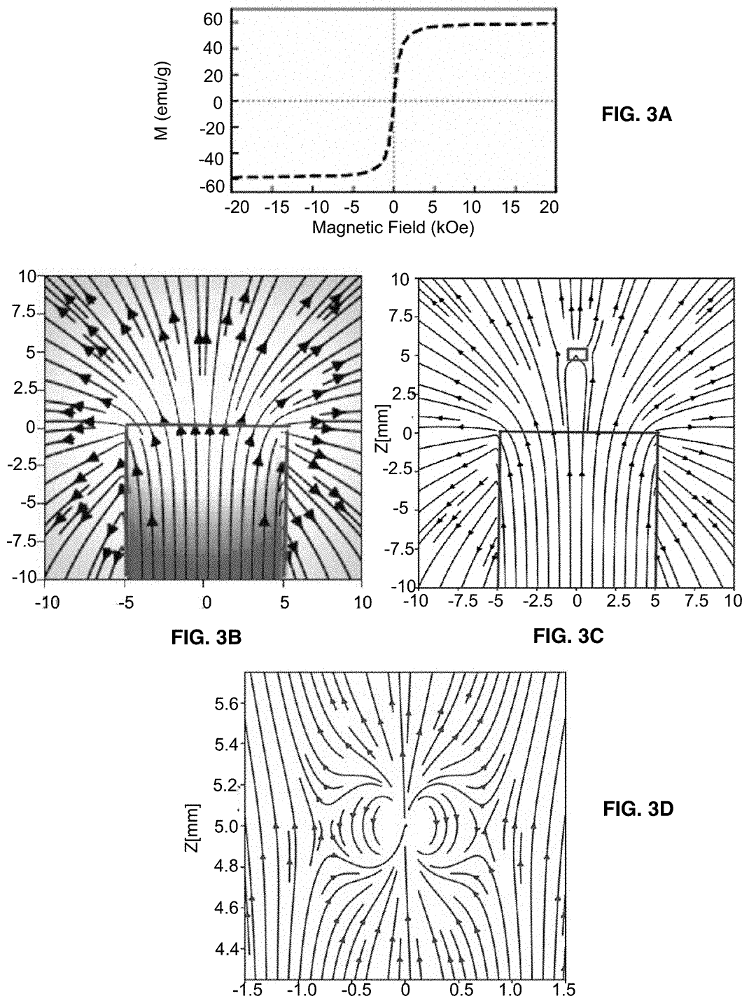

FIG. 3A show the magnetic behavior of the nanoparticles measured at room temperature. The nanoparticles revealed strong magnetization. A mass magnetization (M) of 57-59 emu/g was observed at 5000-20 000 Oe, close to the reported values (80 emu/g). The magnetic particles experience a magnetic force due to a gradient of applied magnetic fields and move toward external magnets. FIG. 3B shows the magnetic field with a gradient around one permanent magnet with a diameter of 10 mm, length of 30 mm, and magnetic field strength of 1 T. The magnetic field is robust, about 932 mT 5 mm away from one pole, and nonuniform, e.g., field gradient is about 7.6 mT/mm along the axis direction of the magnet and 5 mm away from one pole. When one SPIONs nanoparticle is located near the pole, the superparamagnetic iron(II) oxide nanoparticles nanoparticle will be magnetized and rotate itself into alignment with the external magnetic field. At the same time, the local magnetic field will be distorted, as shown in FIG. 3C.

FIG. 3D shows the local magnetic field around a superparamagnetic iron(II) oxide nanoparticles nanoparticle 5 mm away from one pole. The field above 0.5 mm away from the nanoparticle would increase about 10% along the axis direction of the magnet. Under the applied stationary magnetic field and local magnetic gradient, a net magnetic force will drag the magnetic nanoparticles toward the magnet along the direction of the stronger magnetic field. The calculation indicates that the average magnetic force of a superparamagnetic iron(II) oxide nanoparticles nanoparticle with an average diameter of 8.4 nm is about 6.24 Å˜10-20 N (44 times higher than its gravity) when it is axially located at 5 mm above the magnet. Such strong force will drag the nanoparticle toward the magnet efficiently under the magnetic field gradient. The force on individual particles would vary with their location in the magnetic field and their magnetization.

When some superparamagnetic iron(II) oxide nanoparticles exist around one SPION, the superparamagnetic superparamagnetic iron(II) oxide nanoparticles show cooperative magnetophoretic behaviors under the experimental conditions. The permanent magnet and multiple superparamagnetic iron(II) oxide nanoparticles will produce low magnetic gradients, too, close to that of individual SPION (7.6 T/m). The local magnetic gradient is much lower than 100 T/m. According to the magnetophoretic literature, cooperative magnetophoresis would occur and SPIONs would aggregate (29,30). The aggregating superparamagnetic iron(II) oxide nanoparticles would move faster than individual superparamagnetic iron(II) oxide nanoparticles discussed above.

### Fibroblast Cells Viability

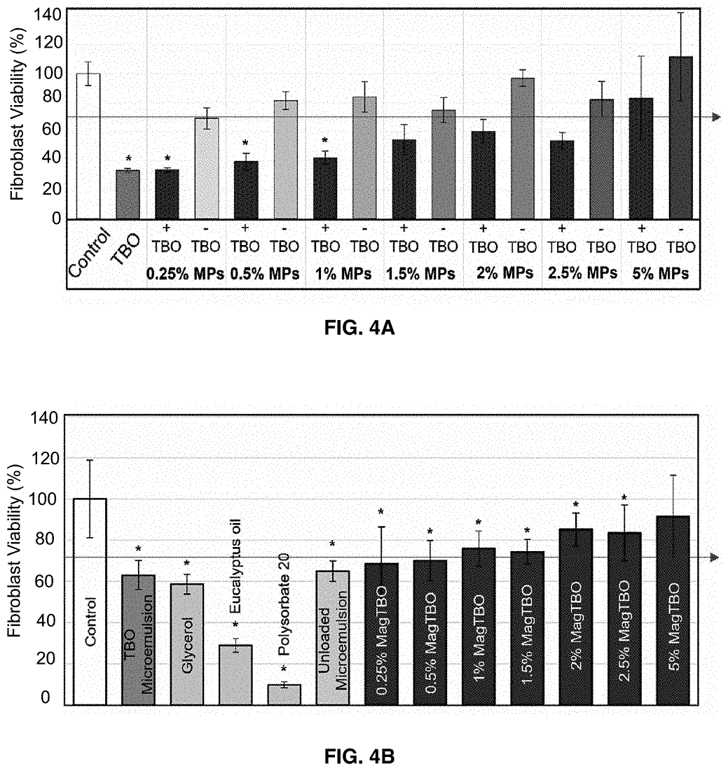

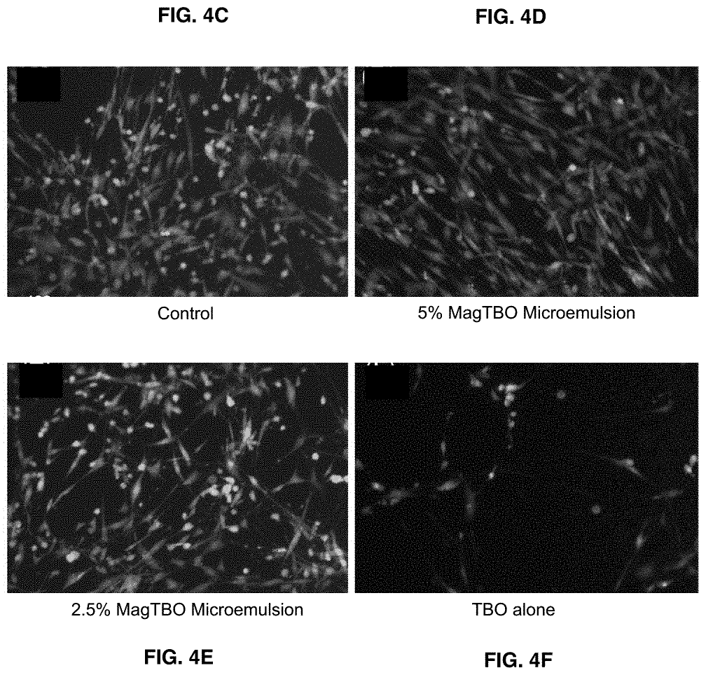

FIG. 4A demonstrates the fibroblast cells viability treated with different concentrations of superparamagnetic iron(II) oxide nanoparticles. All the concentrations (0.25-5%) dissolved in distilled water revealed cell viability higher than 70% compared to the control with no treatment, except for the 0.25% concentration. A positive dose-dependent response is evident as increasing the superparamagnetic iron(II) oxide nanoparticles concentration was associated with more cell viability. The TBO photosensitizer showed significant toxicity compared to the control. When the magnetic nanoparticles were combined with 100 μg/mL TBO in distilled water, only the 5% concentration revealed a viability higher than 70%, indicating that increasing the superparamagnetic iron(II) oxide nanoparticles concentration reduced the cytotoxicity of the TBO. FIG. 4B demonstrates the cytotoxicity of the synthesized microemulsions containing 100 μg/mL TBO and different superparamagnetic iron(II) oxide nanoparticles concentrations. Microemulsions containing superparamagnetic iron(II) oxide nanoparticles with concentrations ranging between 1 and 5% showed a cell viability higher than 70%. Using the 5% MagTBO microemulsion was associated with a cell viability higher than 90%. FIGS. 4C-4F show the live/dead images of the fibroblast cells. The 2.5 and 5% MagTBO microemulsions were associated with a good number of viable cells (FIG. 4D-4E), while using the TBO alone was associated with significantly reduced viability (FIG. 4F).

Encapsulating the TBO inside the microemulsion was also associated with improved TBO's biocompatibility. A previous report showed that 100 μg/mL TBO did not induce significant toxicity against mouse monocyte-macrophage cells (12). However, the results of this study illustrated that TBO alone was very toxic against the human gingival fibroblasts, as the percentage of fibroblast viability was 33.4%. Furthermore, the unloaded microemulsion was associated with a high cytotoxicity against the cells. Accordingly, the cytotoxicity of the microemulsion components was examined. Eucalyptus oil and polysorbate 20 were associated with significant cytotoxicity when added to the fibroblast media in a ratio similar to their proportion in the designed microemulsion. Previous investigations reported the eucalyptus oil's capability to disrupt bacterial membrane (31,32). Therefore, it is highly possible that the contact between eucalyptus oil and gingival fibroblasts cells negatively affected the viability of the cells. However, the cytotoxicity effect of polysorbate 20 against the cells was unexpected. Polysorbate 20 is well-known for being safe and biocompatible (33), and has high safety when injected intramuscularly in animals (34). The polysorbate 20 viscosity, rather than its cytotoxicity, interfered with the cell attachment during the incubation, resulting in less viable cells.

When the TBO was incorporated inside the microemulsion, the viability increased by 1-fold, resulting in more enhanced viability. Such results may encourage higher concentrations of TBO within the microemulsions to maximize the antibacterial reduction, as increasing the concentration of TBO is associated with an enhanced antibiofilm effect (12). Unlike most of the nanoparticles, increasing the superparamagnetic iron(II) oxide nanoparticles concentration was associated with improved biocompatibility. The possible explanation of such findings may be attributed to the capability of superparamagnetic iron(II) oxide nanoparticles in conducting an intrinsic peroxidase-like activity (35,36). Therefore, the cell growth promotion induced by superparamagnetic iron(II) oxide nanoparticles could be achieved by reducing the intracellular H2O2, which may oppose the cytotoxic effect of TBO, eucalyptus oil, and polysorbate 20 (36). Besides, several investigations have shown the positive impact of iron in the cell cycle progression (37,38). It can be concluded that using the microemulsion as a drug carries and the conjugation of superparamagnetic iron(II) oxide nanoparticles reduced the cytotoxicity of the other components within the microemulsion.

### Microemulsion Thermodynamic Stability

Following the fibroblast cells viability assay, three microemulsions were selected for further investigations; 1, 2.5, and 5% MagTBO microemulsions. Table 1 shows that all the microemulsions passed the stress tests as no phase separation was observed. The pH of the microemulsion was very low, indicating a highly acidic solution. Using the spectrophotometer to monitor the absorbance changes, minor differences were observed at 3 and 6 months. The long-term evaluation showed that all the microemulsions were stable for up to six months, whereafter the microemulsions started to show some significant physical changes. The density of the synthesized microemulsions was slightly increased compared to TBO.

TABLE 1
Thermodynamic Stability and the Physical Properties
of the Synthesized MagTBO Microemulsions

| | TBO | 1% MagTBO | 2.5% MagTBO | 5% MagTBO |
|---|---|---|---|---|
| Centrifuge | — | No Phase | No Phase | No Phase |
| Stress Test | | Separation | Separation | Separation |
| Freeze-Thaw | — | No Phase | No Phase | No Phase |
| Stress Test | | Separation | Separation | Separation |
| pH Measurement | 3.19 ± 0.05 | 3.26 ± 0.11 | 3.25 ± 0.15 | 3.31 ± 0.09 |
| Density (g/mL) | 1 | 1.02 | 1.07 | 1.09 |
| Absorbance | | | | |
| change (%) | | | | |
| Baseline | — | — | — | — |
| 1-Month | No change | 0.24 ± 4.93 | 0.17 ± 4.77 | 0.58 ± 1.94 |
| 3-Month | No change | 5.25 ± 3.30 | 2.85 ± 3.95 | 8.44 ± 3.47 |
| 6-Month | No change | 10.29 ± 3.30 | 6.23 ± 3.56 | 13.88 ± 2.70 |
| Long Term | | | | |
| Stability | | | | |
| Baseline | — | No Phase | No Phase | No Phase |
| | | Separation | Separation | Separation |
| 1-Month | — | No Phase | No Phase | No Phase |
| | | Separation | Separation | Separation |
| 3-Month | — | No Phase | No Phase | No Phase |
| | | Separation | Separation | Separation |
| 6-Month | — | No Phase | No Phase | No Phase |
| | | Separation | Separation | Separation |

A variety of nanomaterials have been widely investigated to improve the stability and efficiency of photosensitizers for antimicrobial photodynamic therapy. These approaches involve the use of polymeric nanoparticles as a carrier or functionalizing the photosensitizer into metallic nanoparticles (39). TBO functionalized into chitosan improved the antibacterial action against P. gingivalis, Aggrebacter actinomycetemcomitans, and E. faecalis (40,41). TBO conjugated with several nanoparticles such as gold and silver had demonstrated a more significant antibacterial action compared to the use of TBO alone (42,43). Advanced investigations in antimicrobial photodynamic therapy have also focused on designing and optimizing systems that maximize the photosensitizers' antibacterial effect and improve their stability and biocompatibility, such as nanospheres and emulsions (15).

In the present invention, the TBO photosensitizer was functionalized into superparamagnetic iron(II) oxide nanoparticles inside microemulsions. Microemulsions are metastable colloidal systems with a wide range of applications in medicine and pharmacy (44) and have been used extensively to carry therapeutic agents. Microemulsions are composed of aqueous and organic phases that are dispersed in each other and stabilized by surfactants (emulsifiers) to control the surface tension between the two phases with a polar head and nonpolar tail at the oil-water interface (45). Microemulsions' droplet size ranges from 10 to 100 nm, with a higher thermodynamic stability than nano and macroemulsions, indicating that microemulsions are less likely to experience physical changes over time (46,33).

The designed microemulsions improve the TBO's biocompatibility, as was demonstrated above. Besides biocompatibility, several advantages can be obtained from using microemulsions as a drug delivery approach. Due to their small droplet size, microemulsions provide a good surface area to volume ratio concerning the drug's absorption, thus improving the bioavailability of the loaded drug (47). Besides, droplet size at a small scale increases the resistance of microemulsion against physical changes, resulting in better stability than other emulsions (48). Microemulsions can improve the solubility of poorly water-soluble substances and enhance the biocompatibility of the loaded materials (49,50). In the present invention, the synthesized microemulsions demonstrated good thermodynamic and long-term stabilities responding to different stress challenges and improved the biocompatibility of the TBO photosensitizer.

Antibiofilm Efficacy of MagTBO Platform into the Photodynamic Process

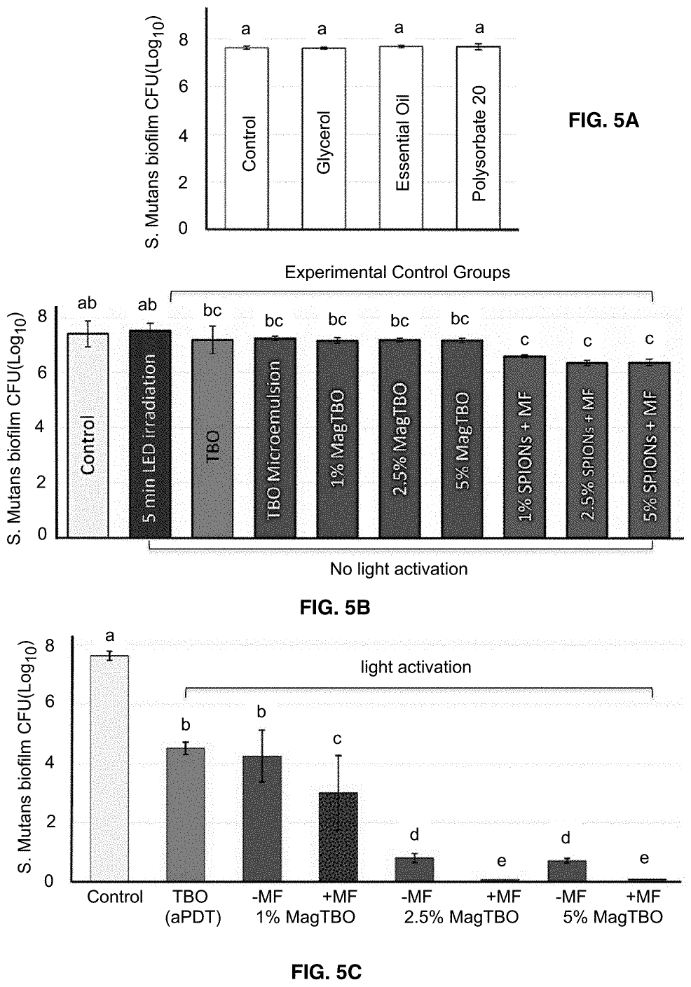

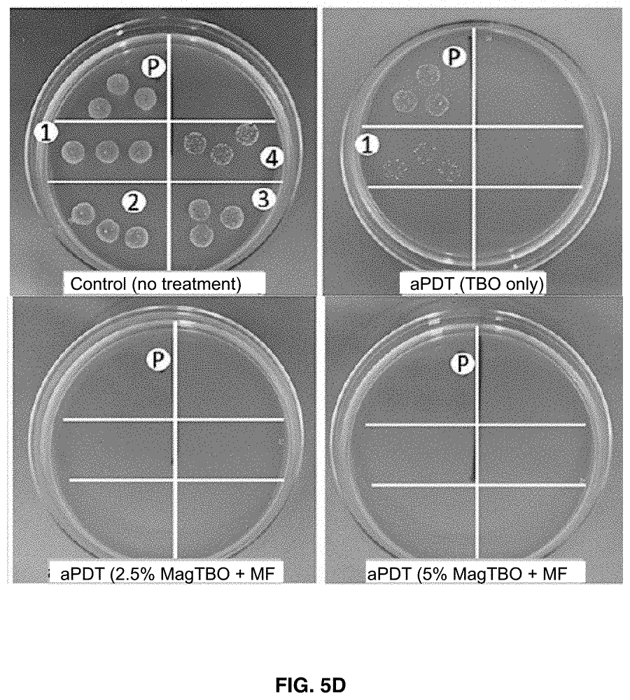

FIG. 5A demonstrates the S. mutans biofilm response to the core components of the synthesized microemulsions, which are glycerol, eucalyptus oil, and polysorbate 20. None of the components were associated with any antibacterial effect, indicating that the antibacterial effect shown in FIG. 5C is dependent on the TBO photosensitizer and the magnetic nanoparticles. No antibacterial effect was observed when the TBO and the synthesized microemulsions were incubated for 5 min without light activation (FIG. 5B). However, the magnetic field applied to the microemulsions containing only SPIONs without the TBO photosensitizer resulted in a 1-log reduction in the S. mutans biofilm (FIG. 5B). Such findings may suggest that the superparamagnetic iron(II) oxide nanoparticles movement under the magnetic field can physically injure the bacterial cells. In FIG. 5C, antimicrobial photodynamic therapy via TBO alone was associated with 3-log reduction (p=<0.05) against the S. mutans biofilm compared to the control (no treatment). Using antimicrobial photodynamic therapy and 1% MagTBO microemulsion resulted in a similar reduction of 3-log. However, when the magnetic field was applied, a 4.5-log reduction was achieved. When the 2.5 and 5% MagTBO microemulsions were photoactivated, a 6-log reduction was observed. Applying the magnetic field with these two concentrations completely eradicated the bacterial biofilm.

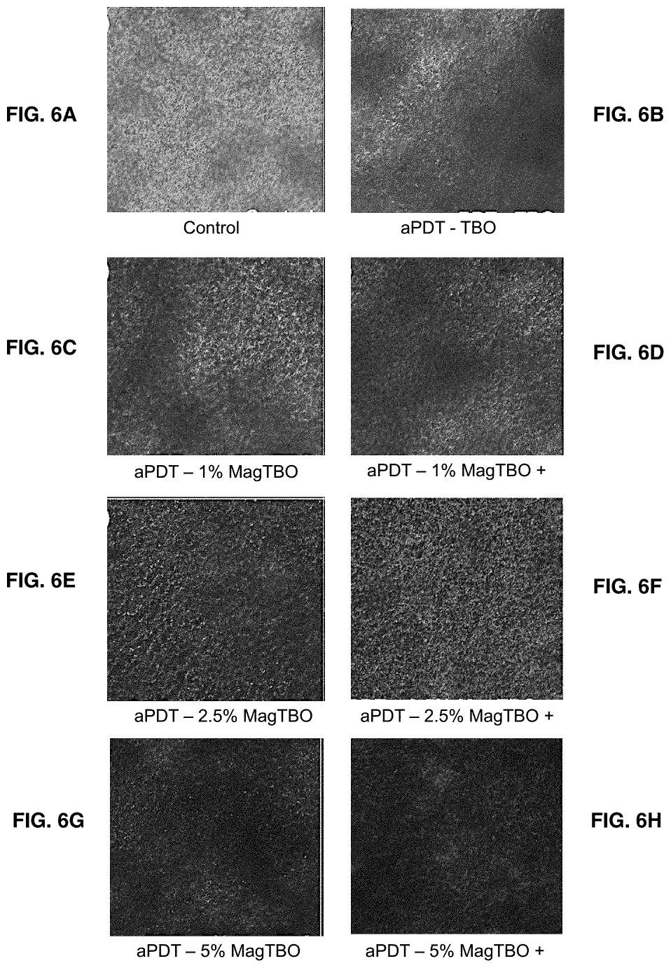

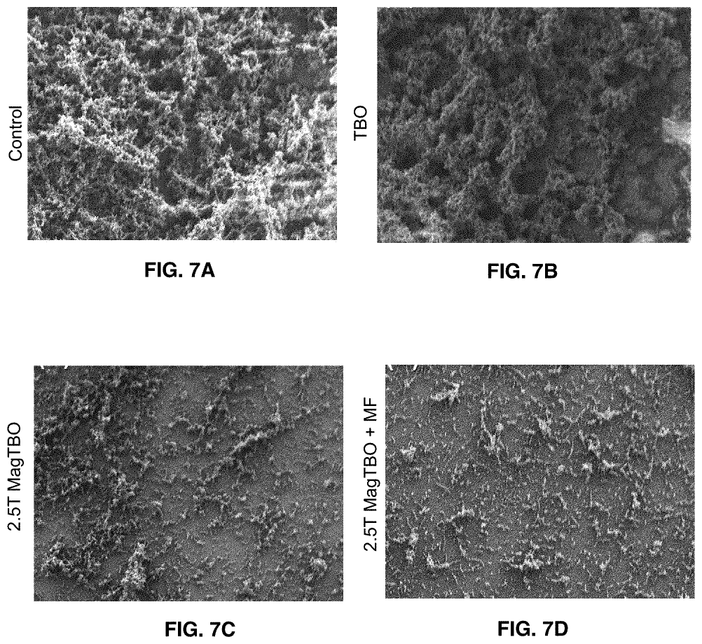

Representative agar plates for the control and different treatment protocols are shown in FIG. 5D. FIGS. 6A-6H illustrate the live/dead images of the S. mutans biofilms subjected to varying types of treatment. A high number of viable colonies can be observed over the control group (FIG. 6A). Antimicrobial photodynamic therapy via TBO alone resulted in a significant reduction in the viable microorganisms (FIG. 6B). Increasing the magnetic concentrations from 1 to 5% and applying the magnetic field (FIGS. 6C-6H) were associated with more dead colonies than the use of TBO alone. The scanning electron microscopy images illustrated that the application of magnetic field force to the 2.5% MagTBO microemulsion (FIG. 7D) was associated with the lower load of S. mutans colonies over the borosilicate glass surface compared to the other groups. Such findings may suggest the benefit of the magnetic field force to damage the biofilm structure resulting in more biofilm inhibition physically.

Reactive oxygen species (ROS) and free radicals in the living cells are kept in balance as they are very destructive when produced in high quantities (51). Photosensitizers with no light activation cannot generate reactive oxygen species, allowing antimicrobial photodynamic therapy to have high specificity for aiming the targeted organs or tissues, one of the main advantages of antimicrobial photodynamic therapy (52). When photosensitizers at a specific wavelength are activated, a high amount of reactive oxygen species is induced. It absorbs the hydrogen found in the cell wall of the targeted cells, which minimizes the cells' wall leading to lysis due to the turgor pressure (53). The use of aPDT has gained much attention recently due to its ability to eradicate bacterial biofilms without inducing bacterial resistance (54). It has been hypothesized that the photo-oxidative damage caused by antimicrobial photodynamic therapy is very aggressive against bacterial biofilms. Thus, the surviving microorganisms are very weak in initiating adaptive mechanisms toward aPDT (55).

In dentistry, antimicrobial photodynamic therapy has been extensively investigated to control several oral diseases, including caries, periodontitis, peri-implantitis, and endodontic infections (10). As a photosensitizer, TBO is a member of the nonporphyrin phenothiazinium family capable of targeting the bacterial membrane and accumulating inside the mitochondria to attack the targeted cells (12). TBO has shown a great antibacterial effect against oral pathogens such as S. mutans, Enterococcus faecalis, and Porphyromonas gingivalis (15). TBO at the concentration of 100 μg/mL was used herein. This concentration did not induce significant toxicity against the macrophage cell line, while concentrations higher than 100 μg/mL were reported with critical cytotoxicity (12).

The clinical outcomes concerning using aPDT in vivo have shown limited effectiveness of less than 2-log reduction (11,13,14). Several photosensitizers' drawbacks may contribute to that, mainly the hydrophobicity of photosensitizers that may limit their penetration through biofilms (56). Besides, photosensitizers are highly susceptible to aggregation when mixed with aqueous solution and prone to degradation due to light sensitivity and elongated storage (57,58). As a result, applying nanotechnology to functionalize photosensitizers may help eliminate these obstacles in antimicrobial photodynamic therapy. The design of nanosystems overcomes the photosensitizers' drawbacks, improves their activity, and enhances biocompatibility (50,59). Most of the reported studies concerning microemulsions aimed to improve the drug delivery to target cancer cells (33). On the contrary, few investigations were conducted to enhance the antimicrobial photodynamic therapy outcomes by incorporating photosensitizers into microemulsions to target pathogenic microorganisms, such as Candida albicans, Escherichia coli, Staphylococcus aureus, and Pseudomonas aeruginosa (60,61).

To robust the antibacterial activities of the TBO-microemulsion, SPIONs were incorporated. Using magnetic nanoparticles is guided by applying a magnetic field force to achieve site-directed drug delivery (62). The most commonly used magnetic nanoparticles in the literature are iron oxide nanoparticles (63). Multiple preclinical and clinical studies have shown the capability of magnetic nanoparticles and magnetic fields in improving the delivery of anticancer drugs to devastate different types of malignant tumors (64).

Magnetic nanoparticles and magnetic fields may induce destructive heat against the targeted cells, inducing apoptosis and cell death (65,66). Moreover, magnetic nanoparticles can internalize inside the targeted cells, allowing the combined therapeutic agents to precisely exert the need for intercellular activity (67). It is contemplated that the magnetic force can direct the particles to biofilms' core, resulting in severe physical damage to the bacterial colonies (68,69).

The effect of the microemulsion containing the superparamagnetic iron oxide nanoparticles without the photosensitizer was examined under the magnetic field force (FIG. 5B). Microemulsions containing only superparamagnetic iron oxide nanoparticles under the magnetic field resulted in around 1-log reduction, which is considered a minor reduction when evaluating the antibacterial properties of dental products. In contrast, the light activation of the MagTBO microemulsions resulted in a massive biofilm inhibition, eradicating the S. mutans biofilm (FIG. 5C).

In antimicrobial photodynamic therapy targeting oral biofilms, encapsulating chlorine e6 (Ce6) and coumarin 6 (C6) with iron oxide magnetic nanoparticles was associated with significant inhibition of 4- to 5-log against periodontal pathogens (70). The present invention combined the microemulsion approach and superparamagnetic iron oxide nanoparticles to improve the antimicrobial photodynamic therapy performance. However, in FIG. 5C, when the CFU of the 1% MagTBO microemulsion with no magnetic force is observed, the bacterial reduction was equivalent to the use of TBO alone, indicating that microemulsion may not positively improve the antimicrobial photodynamic therapy vantibacterial action. In contrast, the application of the magnetic force to the 1% MagTBO microemulsion resulted in more significant bacterial reduction than the TBO alone or the 1% MagTBO microemulsion with no magnetic force. Increasing the magnetic nanoparticles' concentration was associated with a more significant inhibition and applying the magnetic field against biofilms treated with the 2.5 and 5% MagTBO microemulsions completely eradicated the biofilms. In FIG. 5B, incubating the MagTBO microemulsions for 5 min with no light activation did not show antibacterial effects despite the used concentration. However, a dose-dependent antibacterial effect was observed with light activation when the SPIONs concentration was increased from 1 to 2.5 and 5%. Such an observation could be attributed to the ability of SPIONs to produce a high amount of ROS and induce oxidative stress upon radiation (71). Superparamagnetic iron oxide nanoparticles subjected to irradiation therapy had more oxidative stress against cancerous cells than nonirradiated particles (72). It is possible that increasing the concentration may improve the pharmacodynamics of TBO and be associated with a higher adsorption of the TBO inside the targeted cells (73). The present invention suggests three different mechanisms for the potent antibacterial action of the MagTBO microemulsion: (1) ROS generation following the light activation of TBO, (2) the oxidative stress induced by superparamagnetic iron oxide nanoparticles irradiation, and (3) the physical damage caused by the particles and the magnetic field. The massive antibacterial effect was mainly attributed to increasing the concentration of superparamagnetic iron oxide nanoparticles and applying the magnetic field but not microemulsion. However, the use of microemulsion as a carrier is still advantageous. Using the microemulsions may provide more stabilization to the TBO photosensitizer by preventing its degradation and aggregation (33). The small droplet size of the microemulsions encapsulating TBO and superparamagnetic iron oxide nanoparticles together can allow the TBO photosensitizer to be directed to the biofilm's core. It has also been shown that TBO encapsulated inside microemulsion had a greater capability to damage quorum-sensing molecules of the bacterial cells, eliminating the ability of microorganisms to communicate with each other and cooperate to resist external threats (74,75).

Eucalyptus oil was used herein as an organic phase, polysorbate 20 and glycerol as surfactants. Several investigations found that eucalyptus oil has an antimicrobial effect by disrupting microorganisms' cell membranes and allowing the conjugated antibacterial agents to maximize their effect (20,31,32). However, the use of eucalyptus oil alone did not reduce the S. mutans biofilm in this report (FIG. 5A), mainly because the oil was diluted before treating the biofilms to mimic the exact concentration of this oil within the final microemulsion product. Still, it could be possible that eucalyptus oil may hold a synergistic effect when it was added to the microemulsion by making the bacterial cell membrane more vulnerable to aPDT. Polysorbate 20 is one of the most used surfactants due to its stability via forming a self-organized monolayer (76). Polysorbate 20 is a nonionic surfactant used in the food and pharmaceutical industries due to its safety and biocompatibility (33). In microemulsions, surfactants are used in a high percentage to control the tension between the organic and aqueous phases, which explains the high concentration of surfactants used herein (33). As was observed on eucalyptus oil, diluted polysorbate 20 did not show any antibacterial activities against the S. mutans biofilm, indicating that active antibacterial ingredients inside the microemulsions were the TBO photosensitizer and superparamagnetic iron oxide nanoparticles.

### Antibiofilm Efficacy of MagTBO Platform Against Complex Multispecies Biofilms

FIG. 8A illustrates experiments performed following the saliva derived biofilms initiation. After 7 days of biofilm growth over the dentin slabs, samples were subjected to CFU counting, Vicker's microhardness, and confocal and SEM imaging. FIG. 8B demonstrates the findings of the CFUs assay. In general, the use of conventional aPDT via TBO inhibited the multispecies biofilms by around 1-log, compared to the control without treatment yielding nonsignificant reduction (p>0.05). This reduction is less than the S. mutans biofilm inhibition of 3-log, suggesting that the used multispecies model exerts more challenges than the monospecies model. When the 2.5% MagTBO microemulsion was photoactivated against the multispecies biofilms, a significant reduction (p<0.05) of 4 to 4.5-log concerning the total microorganisms, totalstreptococci, mutans streptococci, and total lactobacilli was observed. Applying the magnetic field improved the reduction by 0.5- to 1-log, except for total lactobacilli, compared to the MagTBO with no magnetic field. Even though the magnetic field has improved the 2.5% MagTBO antibiofilm inhibition, the reduction was not statistically significant compared to the 2.5% MagTBO with no magnetic field.

FIG. 8C illustrates the surface microhardness of dentin slabs. Applying the magnetic force would allow the superparamagnetic iron oxide nanoparticles to diffuse inside the dentinal tubules and improve the hardness of the demineralized dentin. However, the surface microhardness was slightly higher than the samples with no treatment.

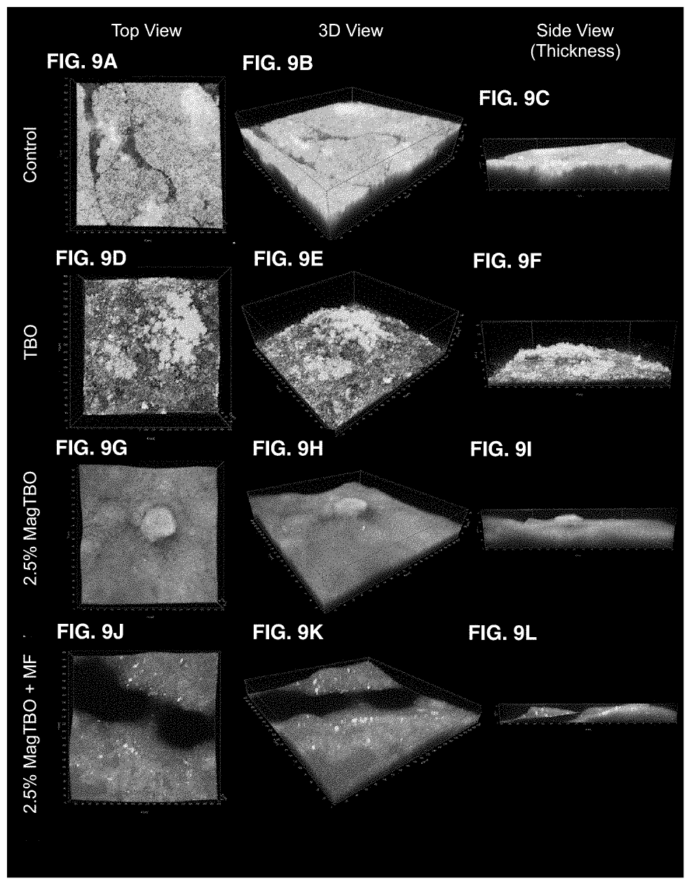

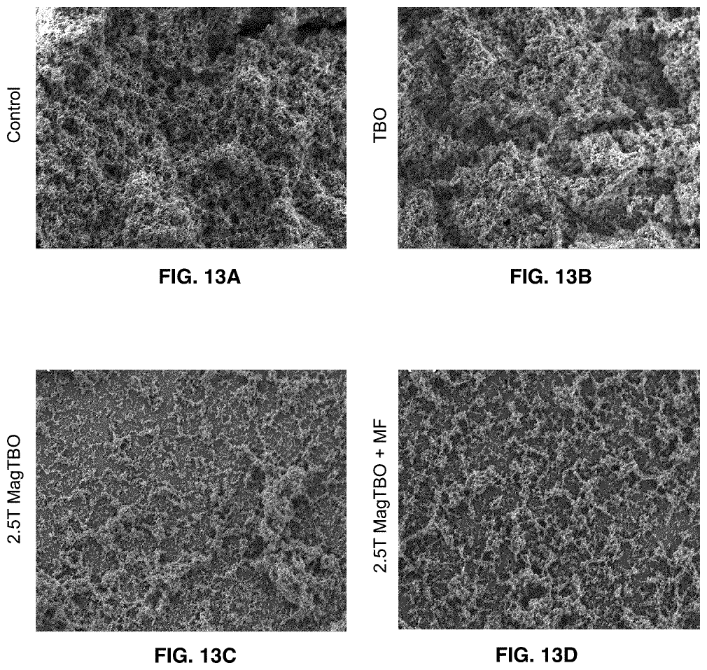

FIGS. 9A-9L and FIGS. 10A-10D support the microbiological outcomes. FIG. 9 illustrates the confocal images of the growing biofilm following each treatment. More viable microorganisms were observed over the samples with no treatment (FIGS. 9A-9C), followed by the samples treated with TBO only (FIGS. 9D-9F). The biofilm thickness values were between 80 and 100 μm in the slabs with no treatment and treated with TBO. Using the 2.5% MagTBO with and without magnetic field demonstrated more areas corresponding to dead/compromised bacteria over the samples (FIGS. 9G-9L) with a biofilm thickness of 60 μm with no magnetic field and 40 μm with the magnetic field. The same pattern can be observed in the SEM images as samples treated with the 2.5% MagTBO microemulsion were associated with less biofilm mass and growth (FIGS. 10A-10D). It has become evident that bacteria in biofilms can provide more aggressive resistance against antimicrobial agents than the planktonic form (77). The biofilm structure and its associated matrix limit the diffusion of antimicrobial agents within the biofilm and protect the embedded bacteria from desiccation and environmental stresses (77). This high level of protection is enhanced in multispecies biofilm compared to monospecies biofilms due to the high synergistic interaction between the species and the capabilities to produce a greater overall biomass (78). As a result, multispecies biofilms demonstrate more resistance against antibiotics and antimicrobial agents than monospecies biofilms (79).

On the basis of these observations, a saliva-derived multispecies biofilm was prepared to investigate the effectiveness of the aPDT via the MagTBO microemulsion against thick and mature multispecies biofilms. Saliva was used as an inoculum to grow the multispecies biofilms and establish a more challenging situation for the MagTBO microemulsion. The use of human saliva as inoculum was demonstrated in several studies to provide more clinically relevant biofilms (80,24,25). The saliva mixture collected from 10 individuals can provide a homogeneous bacterial community and allow for analyzing the growth of different bacterial species. Mutans streptococci and lactobacilli were investigated as they are among the leading pathogens in coronal and root caries (24). Different studies reported that clinically isolated microorganisms might not recover the same quantity and homogeneity when grown in vitro, as several species could be lost (80,26). Therefore, such a model cannot be relied on to resemble the oral cavity's microbiota exactly. The same issue is applied when discussing the selective agar media used to isolate and enumerate the bacterial species (81).

The following references are cited herein.
1. Flemmig, T. F.; Beikler, T. Periodontol 2000 2011, 55(1):9-15.
2. Jiao, Y.; Tay, F. R.; Niu, L.; Chen, J. Int. J. Oral Sci. 2019, 11(3):1-11.
3. Radaic, et al. Comput. Struct. Biotechnol. J. 2021, 19:1335-1360.
4. Bowen, et al. Trends Microbiol. 2018, 26 (3):229-242.
5. de Melo, et al. Expert Rev. Anti-Infect. Ther. 2013, 11 (7):669-693.
6. Muhammad, et al. Front. Microbiol. 2020, 11:928.
7. Pinto, et al. Front. Microbiol. 2020, 11:952.
8. Cieplik, et al. Front. Microbiol. 2014, 5:405.
9. Teófilo, et al. Photodiagn. Photodyn. Ther. 2021, 33:102046.
10. Mang, et al. Lasers Surg. Med. 2012, 44(7):588-596.
11. Melo, et al. Photodiagn. Photodyn. Ther. 2015, 12(4):581-586.
12. Balhaddad, et al. Int. J. Mol. Sci. 2020, 21(20):7612.
13. Guglielmi, et al. J. Biomed. Opt. 2011, 16(8):088003.
14. Araujo, et al. Photodiagn. Photodyn. Ther. 2015, 12(3):401-407.
15. Balhaddad, et al. Photobiomodul Photomed Laser Surg 2020, 38:481.
16. Mitwalli, et al. Bioengineering (Basel) 2020, 7(3):83.
17 75. Sun, et al. ACS Appl. Mater. Interfaces 2017, 9(13):11451-11460.
18 76. Bhadila, et al. J. Dent. 2020, 99:103406.
19 18. Leanpolchareanchai, et al. Molecules 2014, 19(11):17107-17129.
20. Rout, et al. Colloids Surf. B 2016, 140:472-480.
21. Badawi, et al. AAPS PharmSciTech 2009, 10(4):1081-1084.
22. Shao, et al. Sci. Rep. 2018, 8(1):10565.
23. Gomez, et al. Curr. Microbiol. 2016, 73(3):426-433.
24. Balhaddad, A et al. Dent. Mater. 2020, 36(8):e266-e278.
25. Mitwalli, et al. J. Funct Biomater. 2020, 11(3):56.
26. Ibrahim, et al. Biomater. Sci. 2020, 8(12):3472-3484.
27. Bhadila, et al. Acta Biomater. 2020, 114:146-157.
28. McBain, A. J. Chapter 4 In Vitro Biofilm Models: An Overview. In Advances in Applied Microbiology; Academic Press, New York, N.Y., 2009; Vol. 69, pp 99-132.
29. Leong, et al. Langmuir 2020, 36 (28):8033-8055.
30. Faraudo, et al. Colloid Polym. Sci. 2010, 288(2):207-215.
31. Clavijo-Romero, et al Food Sci. Technol. Int. 2019, 25(1):24-37.
32. Bakkali, et al. Food Chem. Toxicol. 2008, 46(2):446-475.
33. Callender, et al. Int. J. Pharm. 2017, 526(1):425-442.
34. Kim, et al. PLoS One 2021, 16(8):e0256869.
35. Gao, et al. Nat. Nanotechnol. 2007, 2(9):577-583.
36. Huang, et al. Biomaterials 2009, 30(22):3645-3651.
37. Chaston, et al. Clin. Cancer Res. 2003, 9(1):402-414.
38. Simonart, et al. J. Invest. Dermatol. 2000, 115(5):893-900.
39. Sobczyński, J. and Polski, A. Chapter 21—Nanocarriers for Photosensitizers for Use in Antimicrobial Photodynamic Therapy. In Nanostructures for Antimicrobial Therapy; Ficai, A., Grumezescu, A. M., Eds.; Micro and Nano Technologies; Elsevier, Atlanta, Ga., 2017; pp 481-502.
40. Peng, et al. Int. J. Mol. Sci. 2016, 17(11):1821.
41. Camacho-Alonso, et al. Photomed Laser Surg 2017, 35(4):184-189.
42. Narband, et al. Current Nanoscience 2008, 4(4):409-414.
43. Misba, et al. Biofouling 2016, 32(3):313-328.
44. Gibaud, S. and Attivi, D. Expert Opin. Drug Delivery 2012, 9(8):937-951.
45. Müllertz, et al. J. Pharm. Pharmacol. 2010, 62(11):1622-1636.
46. McClements, D. J. Soft Matter 2012, 8(6):1719-1729.
47. Liu, et al. J. Drug Target 2016, 24 (8):694-702.
48. Kamboj, et al. Eur. J. Pharm. Sci. 2015, 78:90-102.
49. Jambhekar, S. S. and Breen, P. J. Drug Discovery Today 2013, 18(23-24):1173-1184.
50. Feng, et al. Colloids Surf. B 2015, 125:197-205.
51. Vatansever, et al. FEMS Microbiology Reviews 2013, 37(6):955-989.
52. Alfirdous, et al. Appl. Sci. 2021, 11 (11), 4759.
53. Diogo, et al. J. Funct Biomater. 2019, 10 (4), 44.
54. Hamblin, M. R.; Hasan, T. Photochem. Photobiol. Sci. 2004, 3(5):436-450.
55. Liu, et al. J. Clin Transl Res. 2015, 1 (3), 140-167.
56. Melo, M. A. S. Photomed. Laser Surg. 2014, 32 (11):589-591.
57. Rossetti, et al. J. Controlled Release 2011, 155(3):400-408.
58. Burkhard, N. and Guth, J. A. Pestic. Sci. 1976, 7(1):65-71.
59. Feng, et al. Colloids Surf., B 2013, 107:220-226.
60. Rocha, et al. Photodiagn. Photodyn. Ther. 2020, 32:101962.
61. Liu, et al. RSC Adv. 2016, 6(67):63013-63022.
62. Alexiou, et al. J. Magn. Magn. Mater. 2011, 323(10):1404-1407.
63. Tietze, et al. Biochem. Biophys. Res. Commun. 2015, 468(3):463-470.
64. Huang, et al. Adv. Funct. Mater. 2016, 26(22):3818-3836.
65. Kale, et al. Cell Death Differ. 2018, 25(1):65-80.
66. Dorniani, et al. Int. J. Nanomed. 2012, 7:5745-5756.
67. Sulaiman, et al. Artif. Cells, Nanomed., Biotechnol. 2018, 46(6):1215-1229.
68. Li, et al. Nanoscale 2019, 11(14):6905-6915.
69. Ng, et al. Langmuir 2020, 36:5085.
70. Sun, et al. J. Dent. 2019, 81:70-84.
71. Aranda, et al. Toxicol. In Vitro 2013, 27(2):954-963.
72. Hauser, et al. Biomaterials 2016, 105:127-135.
73. Al-Rawi, et al. Saudi Pharm. J. 2020, 28(7):876-887.
74. Rout, et al. Photodiagn. Photodyn. Ther. 2018, 21:190-200.
75. Parsek, M. R. and Greenberg, E. P. Trends Microbiol. 2005, 13(1):27-33.
76. Shen, et al. Surf. Sci. 2011, 605 (5):494-499.
77. Flemming, H.-C. and Wingender, J. Nat. Rev. Microbiol. 2010, 8(9):623-633.
78. Burmølle, et al. Appl. Environ. Microbiol. 2006, 72(6):3916-3923.
79. Røder, et al. Trends Microbiol. 2016, 24(6):503-513.
80. Balhaddad, et al. J. Dent Res. 2021, 100:1063-1071.
81. Bonnet, et al. New Microbes New Infect. 2020, 34:100622.

What is claimed:

1. An antimicrobial microemulsion, comprising:
a plurality of superparamagnetic iron oxide nanoparticles; and
a photosensitizer in a functional association with the plurality of superparamagnetic iron oxide nanoparticles.

2. The antimicrobial microemulsion of claim 1, wherein the superparamagnetic iron oxide nanoparticles are superparamagnetic iron(II) oxide nanoparticles.

3. The antimicrobial microemulsion of claim 1, wherein the photosensitizer is toluidine blue O.

4. The antimicrobial microemulsion of claim 1, wherein the antimicrobial microemulsion comprises a magnetic-responsive photodynamic nanoplatform configured for targeted delivery of the plurality of superparamagnetic iron oxide nanoparticles and the photosensitizer.

5. A pharmaceutical composition comprising the antimicrobial microemulsion of claim 1.

6. A method for treating an oral disease in a subject in need thereof, comprising:
applying the antimicrobial microemulsion of claim 1 to an oral area of interest in the subject;
irradiating the plurality of superparamagnetic iron oxide nanoparticles and the photosensitizer in the functional association therewith during an application of a magnetic field to the superparamagnetic iron oxide nanoparticles; and
targeting, via the magnetic field, the plurality of superparamagnetic iron oxide nanoparticles and the photosensitizer to the oral area of interest, thereby treating the oral disease in the subject.

7. The method of claim 6, wherein the irradiating step comprises applying an antimicrobial photodynamic therapy.

8. The method of claim 6, wherein the oral disease is selected from the group consisting of at least one of caries, periodontitis, peri-implantitis, and an endodontic infection.

9. A magnetic-responsive photodynamic nanoplatform, comprising:
in a microemulsion:
a plurality of superparamagnetic iron(II) oxide nanoparticles; and
an organic photosensitizer functionally associated with the plurality of superparamagnetic iron oxide nanoparticles.

10. The magnetic-responsive photodynamic nanoplatform of claim 9, wherein the organic photosensitizer is toluidine blue O (TBO).

11. An process for decreasing a microbial population in a pathogenic oral biofilm, comprising:
contacting the oral biofilm with the microemulsion comprising the magnetic-responsive photodynamic nanoplatform of claim 9;
applying a photodynamic therapy to the plurality of superparamagnetic iron oxide nanoparticles and the organic photosensitizer functionally associated therewith in the microemulsion; and
applying a magnetic field to the superparamagnetic iron oxide nanoparticles during the photodynamic therapy to target the plurality of superparamagnetic iron oxide nanoparticles and the organic photosensitizer to the microbial population of the pathogenic oral biofilm, said the plurality of superparamagnetic iron oxide nanoparticles and the organic photosensitizer pharmacologically effective to decrease the microbial population therein.

12. The process of claim 9, wherein the step of applying the photodynamic therapy comprises irradiating the plurality of superparamagnetic iron oxide nanoparticles and the organic photosensitizer functionally associated therewith.

13. The process of claim 9, wherein the photodynamic therapy is an antimicrobial photodynamic therapy.

14. The process of claim 9, wherein the microbial population in the pathogenic oral biofilm is a single pathogenic species biofilm or a multispecies pathogenic biofilm.

15. The process of claim 9, wherein the microbial population comprises at least one oral pathogenic microbe selected from the group consisting of Streptococcus mutans, Enterococcus faecalis, and Porphyromonas gingivalis.

16. A method for improving the efficacy of a photosensitizer during an antimicrobial photodynamic therapy treatment of an oral disease, comprising:
encapsulating a photosensitizer and a plurality of superparamagnetic iron(II) oxide nanoparticles in a microemulsion whereby the photosensitizer is functionally associated with the plurality of superparamagnetic iron oxide nanoparticles; and
irradiating the photosensitizer and the superparamagnetic iron oxide nanoparticles to activate the same during an application of a magnetic field thereto, whereby the magnetic field directs the superparamagnetic iron oxide nanoparticles with the functionally associated photosensitizer to a pathogenic microbe causing the oral disease, thereby improving the efficacy of the photosensitizer during the aPDT treatment of the oral disease.

17. The method of claim 16, wherein the microemulsion encapsulating the superparamagnetic iron oxide nanoparticles and the photosensitizer functionally associated therewith comprise a photodynamic nanoplatform.

18. The method of claim 16, wherein the photosensitizer is toluidine blue 0.

19. The method of claim 16, wherein the oral disease is selected from the group consisting of at least one of caries, periodontitis, peri-implantitis, and an endodontic infection.

20. The method of claim 16, wherein the pathogenic microbe is selected from the group consisting of at least one of Streptococcus mutans, Enterococcus faecalis, and Porphyromonas gingivalis.
